(19) United States
(12) Patent Application Publication
(10) Pub. No.: US 2008/0188872 A1
(43) Pub. Date: Aug. 7, 2008

(54) Multi-mode clip applier, and associated method

(76) Inventor: Michael Duff, Osage Beach, MO (US)

Correspondence Address: LATHROP & GAGE LC, 1845 S. NATIONAL, P.O. BOX 4288, SPRINGFIELD, MO, 65101, US

(21) Appl. No.: 12/001,822

(22) Filed: Dec. 13, 2007

Related U.S. Application Data
(60) Provisional application No. 60/874,760, filed on Dec. 13, 2006.

(51) Int. Cl. A61B 17/10

(52) U.S. Cl. 606/142

(57) ABSTRACT

A multi-mode clip applier includes a hand-held applier body having a handle and a trigger, and internal mechanisms for firing a surgical clip upon actuation of the trigger. Distal jaws close when actuated by the internal firing mechanisms. An open-mode switch limits range of motion of the internal firing mechanisms and closure of the distal jaws, to produce open-mode surgical clips. Actuating the trigger when the open-mode switch is engaged drives the internal firing mechanisms through their limited range of motion to limit clamping of the distal jaws, bend free clip ends together, circumferentially bounding a clipped structure. A release button releasing the open-mode switch, to produce a closed surgical clip when the applier is fired.

### RELATED APPLICATIONS

This application claims priority to U.S. Provisional Patent Application Ser. No. 60/874,760, filed Dec. 13, 2006 and incorporated herein by reference.

### BACKGROUND

In modem surgery, it is often necessary to clip blood vessels, tubular organs and other internal body structures, whether to mark a tumor for radiologic tracking or to ligate a vessel or duct prior to dissection. For example, cholecystectomy, the treatment of choice for symptomatic gallstones, has advanced from an open surgery to a widely-used laparoscopic procedure utilizing surgical clips to ligate the cystic duct and artery.

A surgeon performing laparoscopic cholecystectomy, or gallbladder removal, generally utilizes surgical clips to ligate the cystic artery and the cystic duct, before dissecting the gallbladder from the liver bed. In many institutions, a routine operative cholangiogram is also performed, to image the gallbladder, the cystic duct or the biliary tree, for example to aid the surgeon in differentiating the cystic duct from the common bile duct, to avoid accidental dissection of the latter. Commonly, interoperative cholangiogram includes the injection of saline or high-contrast fluids into the structure or system of interest, followed by imaging.

Recent years have seen considerable advances in laparoscopic and endoscopic procedures such as cholecystectomy/cholangiography. Many of these advances stem from the increasing versatility of endoscopic staple and clip applying devices. These devices are generally advanced through the cannula of an endoscopic trocar, to tissue that is to be clipped or ligated.

As laparoscopic/endoscopic tools have advanced, inadequacies have become increasingly apparent. For example, conventional applying devices may injure clipped ducts, vessels or other tubular structures. Prior art clip appliers that are intended for use in ligating a structure prior to dissection typically close clips by flattening them tightly onto the structure. Because they aim to securely and, for the most part, permanently seal tubular structures (for example to prevent excess bleeding or leakage of ductile fluids into the body cavity) clips applied by prior art appliers are often difficult to remove if misplaced. In addition, even if misplaced clips are discovered and successfully removed, they may damage the mistakenly clipped structure. For example, damage to the common bile or common hepatic ducts (e.g., by erroneously clipping these structures) is recognized as a serious risk and complication in cholecystectomy. Clips placed upon biliary structures such as the common bile duct may compromise patient health, for example lacerating the duct, reducing blood flow to the duct or predisposing the duct to stricture formation after the misplaced clip is removed. Stricture formation may in turn result in stagnant bile flow, leading to cirrhosis and even liver failure. For such reasons, prior art appliers preclude the use of clips/staples as markers.

### SUMMARY OF THE INVENTION

The multi-mode clip applier and associated method of use disclosed herein may reduce injury to mis-clipped structures (hereinafter interchangeably referred to as “ducts”, “tubular structures” and “vessels”), and additionally advance use of clips as markers in imaging procedures such as cholangiography and tumor and aneurysm visualization.

In one embodiment, a multi-mode clip applier includes a hand-held applier body having a handle and a trigger; internal mechanisms for firing a surgical clip upon actuation of the trigger, and distal jaws configured to close when actuated by the internal firing mechanisms. An open-mode switch limits range of motion of the internal firing mechanisms and closure of the distal jaws, to produce open-mode surgical clips. A release button releases the open-mode switch, to produce closed surgical clips upon actuating the trigger. Actuating the trigger when the open-mode switch is released drives the internal firing mechanisms through their full range of motion and closes the distal jaws to a least-separated position, to flatten the surgical clip.

In one embodiment, a method for multi-mode surgical clipping includes selecting an open clipping mode on a multi-mode clip applier; advancing distal jaws of the multi-mode clip applier to a body structure, and clipping the structure with an open-mode clip, to circumferentially bound the structure. Correct placement of the open-mode clip is verified, and a closed clipping mode is selected on the multi-mode clip applier, when the placement of the open-mode clip is correct. When the placement of the open-mode clip is correct, the clip is flattened into closed mode with the multi-mode clip applier.

### BRIEF DESCRIPTION OF THE FIGURES

FIG. 1 is a side view of a prior art surgical clip.

FIG. 2 is a side view showing the clip of FIG. 1, as closed by a prior art clip applier.

FIG. 3 is a partial side view of the clip of FIG. 1 within the jaws of a prior art clip applier.

FIG. 4 is a side view illustrating flattening of the clip of FIG. 1 by the prior art applier jaws of FIG. 3.

FIGS. 5-7 are partial side views of the prior art applier of FIG. 3, prior to and after firing a surgical clip.

FIG. 8 is a partial, schematic side view of a hand-held portion of a multi-mode clip applier for multi-mode closure of surgical clips, with an engaged switch for producing open-mode clips, in accord with an embodiment.

FIG. 9 is a side view of a surgical clip in open mode, as produced by the multi-mode clip applier of FIG. 8, in accord with an embodiment.

FIG. 10 is a partial, schematic side view showing the open mode clip of FIG. 9, within the jaws of the multi-mode clip applier of FIG. 8.

FIG. 11 is a perspective view, showing exemplary features of the multi-mode clip applier jaws of FIG. 10, in accord with an embodiment.

FIG. 12 is another perspective view showing further exemplary features of the clip applier jaws of FIG. 10, in accord with an embodiment.

FIG. 13 is also a perspective view, illustrating additional exemplary features of the clip applier jaws of FIG. 10, in accord with an embodiment.

FIG. 14 is a partial, schematic side view of the hand-held portion of the multi-mode clip applier of FIG. 8 prior to firing, with the open-mode switch disengaged, in accord with an embodiment.

FIG. 15 is a partial side view of the hand-held portion of FIG. 14 after firing, in accord with an embodiment.

FIG. 16 is a schematic side view depicting the multi-mode clip applier jaws and clip of FIG. 10, showing the positioning of the clip around a duct or vessel, in accord with an embodiment.

FIG. 17 is a schematic side view of the multi-mode clip applier jaws and clip of FIG. 10, showing application of the clip in open mode around the duct or vessel of FIG. 16, in accord with an embodiment.

FIG. 18 is a schematic side view depicting the multi-mode clip applier jaws and clip of FIG. 10, the clip flattened around the vessel of FIG. 16, in accord with an embodiment.

FIG. 19 is a schematic side view of the multi-mode clip applier of FIGS. 8, 14 and 15 with a jaw-actuating cannula, in accord with an embodiment.

FIG. 20 is a schematic side view of the multi-mode clip applier of FIGS. 8, 14 and 15 with a jaw-actuating wire, in accord with an embodiment.

FIG. 21 is a schematic side view of the multi-mode clip applier jaws of FIGS. 10-13 and 16-18, with dimensions, in accord with an embodiment.

FIG. 22 is a flowchart illustrating a method for multi-mode clipping, in accord with an embodiment.

### DETAILED DESCRIPTION OF THE FIGURES

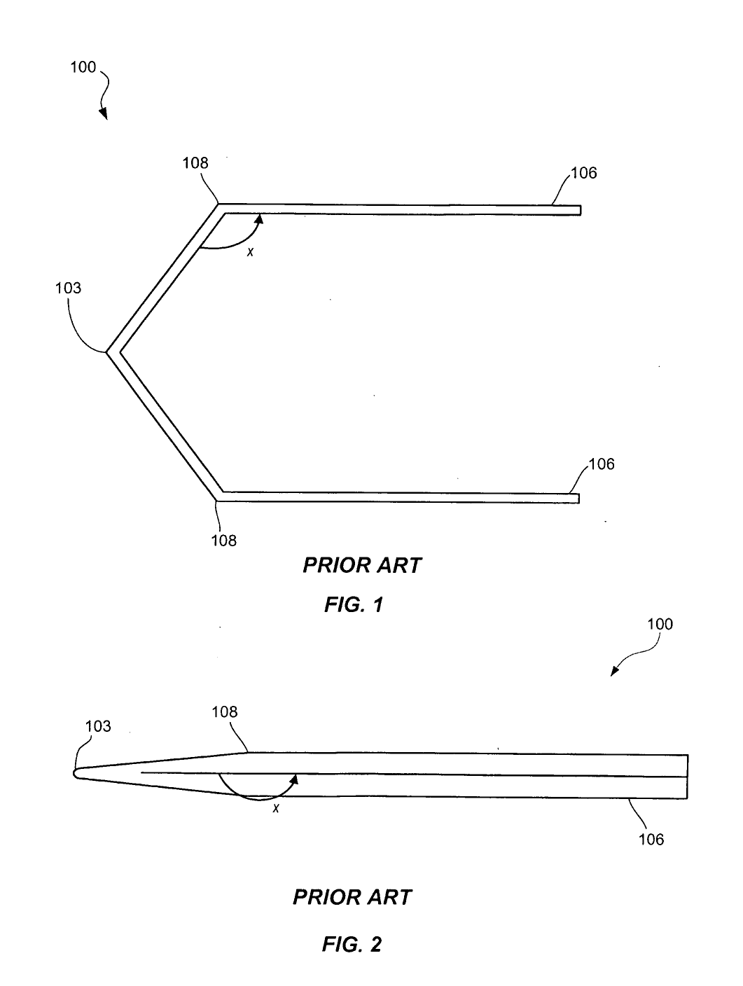

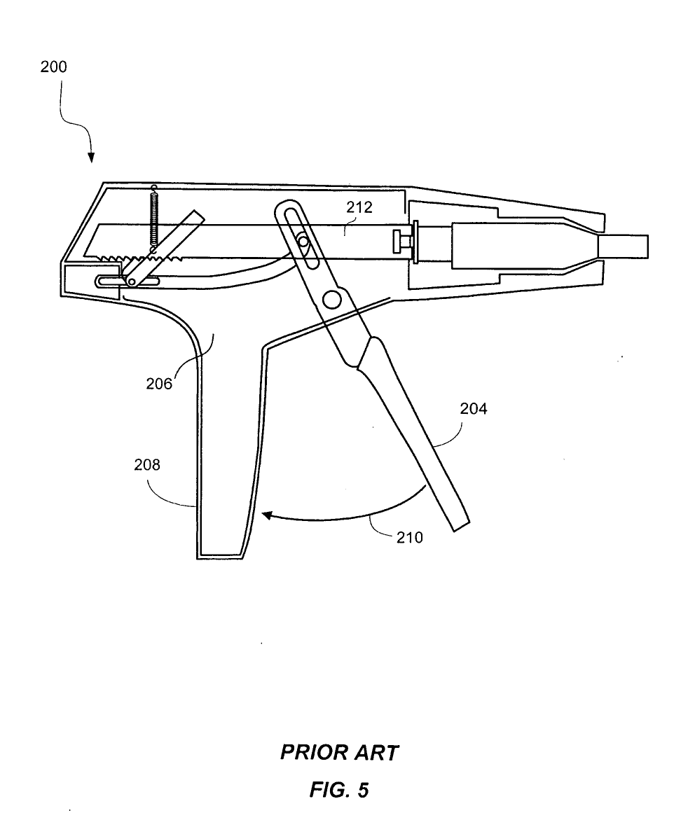

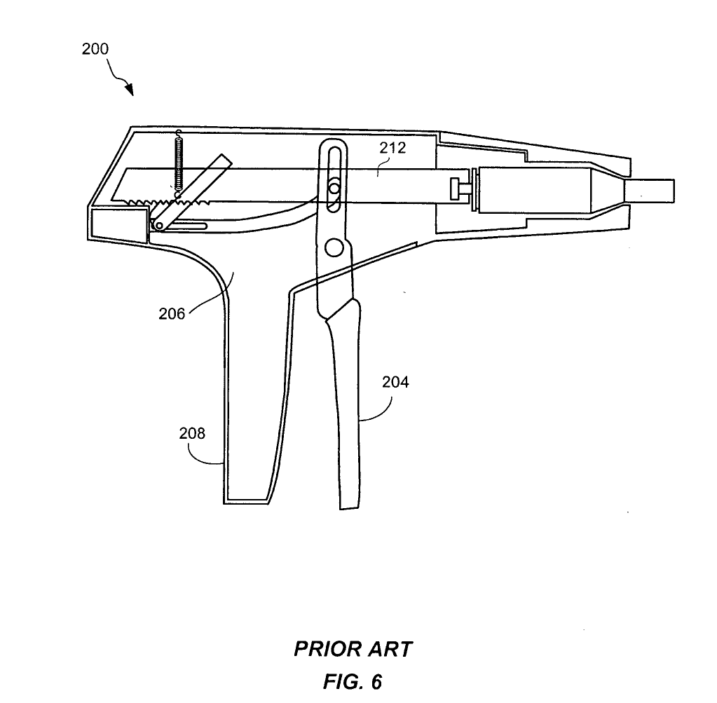

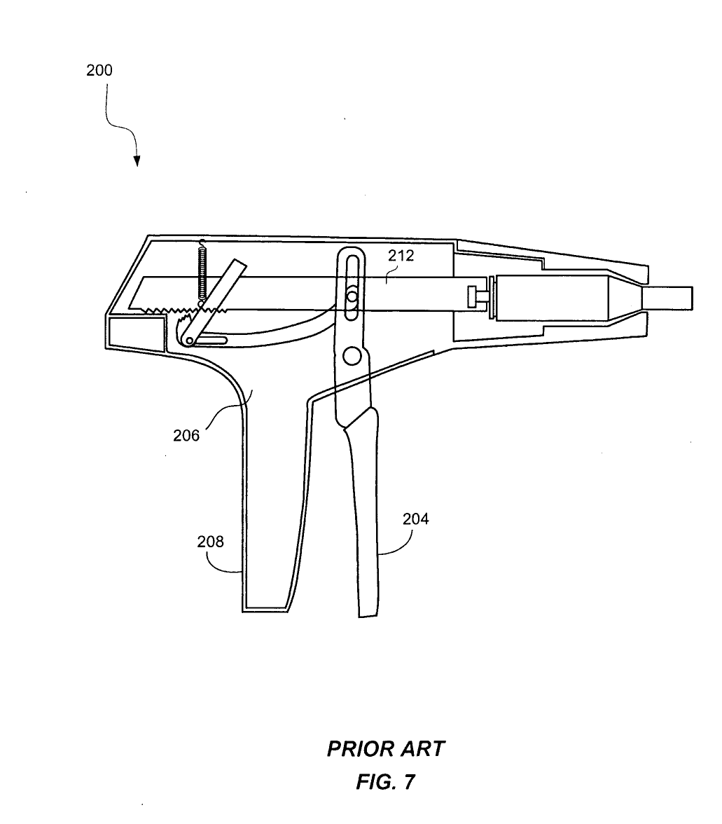

FIGS. 1-4 schematically illustrate a prior art clip or staple 100 (hereinafter, “clips” and “staples” may be used interchangeably) and a prior art clip applier 200. In particular, FIG. 1 shows clip 100 in its manufactured, un-clipped form, and FIG. 2 shows clip 100 as flattened and applied by a prior art applier (see, e.g., FIGS. 5-7). Clip 100 has an apex or nose 103, arms 106 and shoulders 108 between nose 103 and arms 106. When flattened by a prior-art applier, angle χ at shoulders 108 is increased as shoulders 108 are flattened into alignment with arms 106 and nose 103. Angle χ is shown as approximately 180° at FIG. 2, to emphasize flattening of clip 100 by a prior-art applier. The actual increase in angle χ when clip 100 is flattened may vary according to the prior-art applier used.

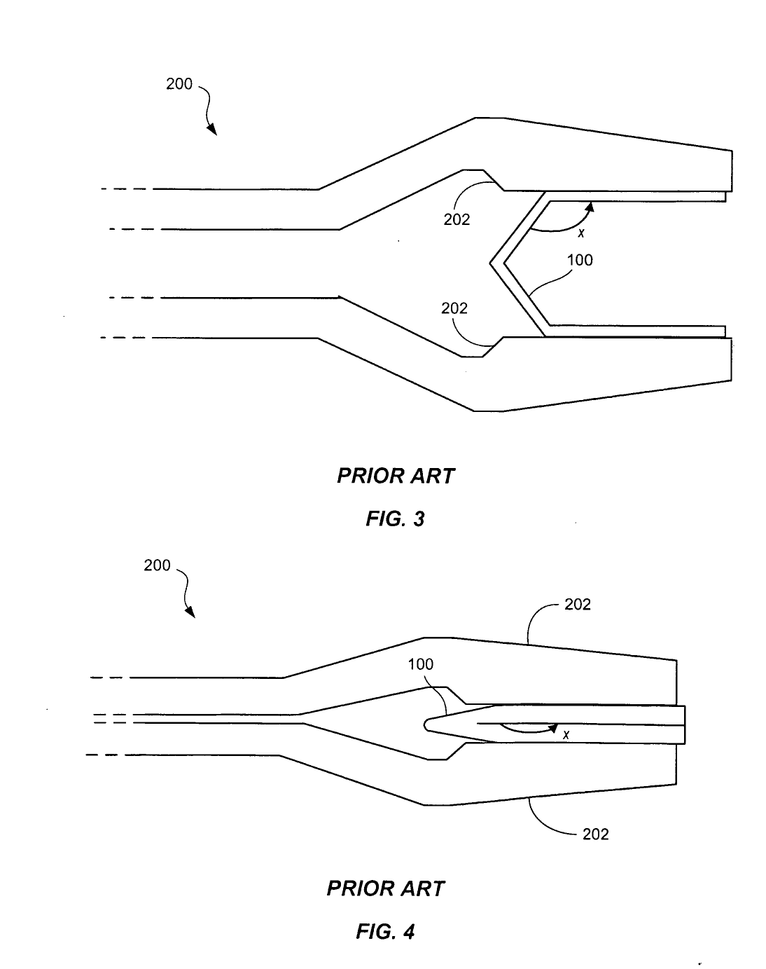

FIG. 3 shows clip 100 grasped within jaws 202 of a prior art applier 200. Jaws 202 close to compress clip 100 into its flattened state, increasing angle χ as shown in FIG. 4. Jaws 202 may be closed remotely by applying pressure to a trigger, such as trigger 204, shown in FIGS. 5-7.

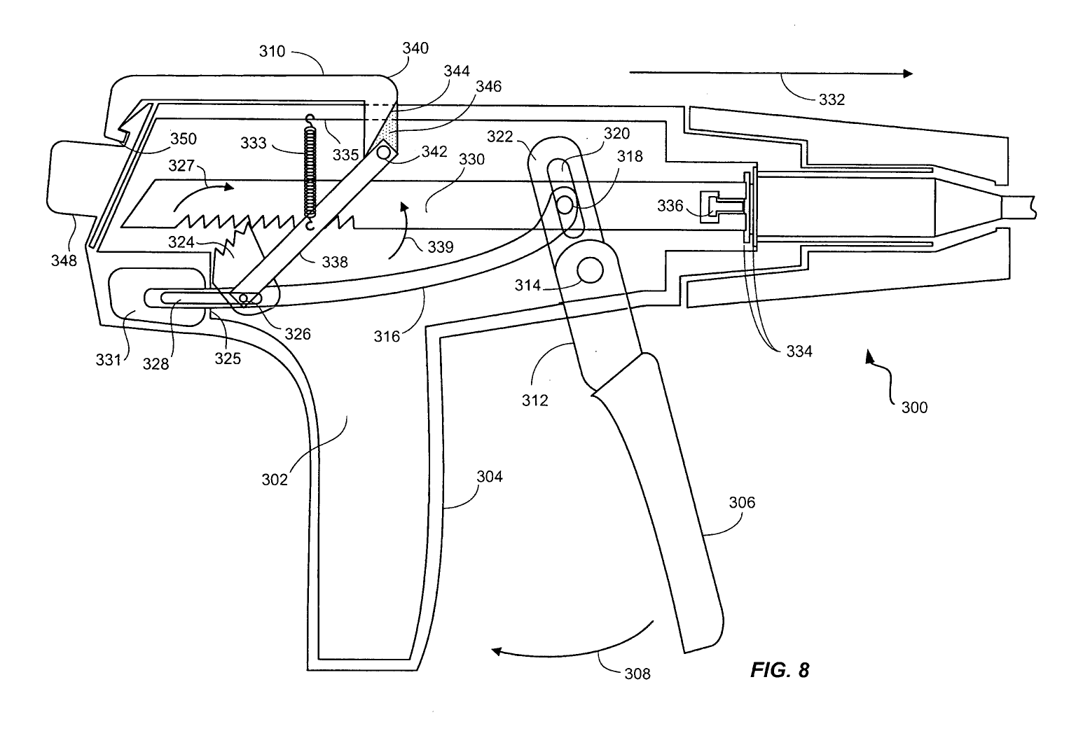

FIGS. 5-7 provide a partial view of prior art applier 200, in particular, schematically showing a hand-held portion 206. A surgeon or practitioner for example grasps trigger 204 with the fingers while securing handle 208 in the crook of the thumb and against the palm. Pulling trigger 204 toward handle 208 (as indicated by arrow 210) advances a plate 212 and actuates a mechanism to close jaws 202 (see FIGS. 3-4) and flatten clip 100. Jaws 202 are for example connected to hand-held portion 206 (positioned outside of a patient's body) via support tubes extending through the cannula of an endoscopic trocar into the patient's body, through a laparoscopy site. Exemplary prior art appliers are described in the following U.S. Patents, which are incorporated herein by reference: U.S. Pat. No. 5,171,249, issued to Stefanchik; U.S. Pat. No. 6,843,794, issued to Sixto, Jr. et al., and U.S. Pat. No. 5,100,420, issued to Green et al. These prior art appliers do not provide for temporary or circumferential clipping of tubular structures (e.g., “open mode” clipping, described below with respect to FIGS. 8-10).

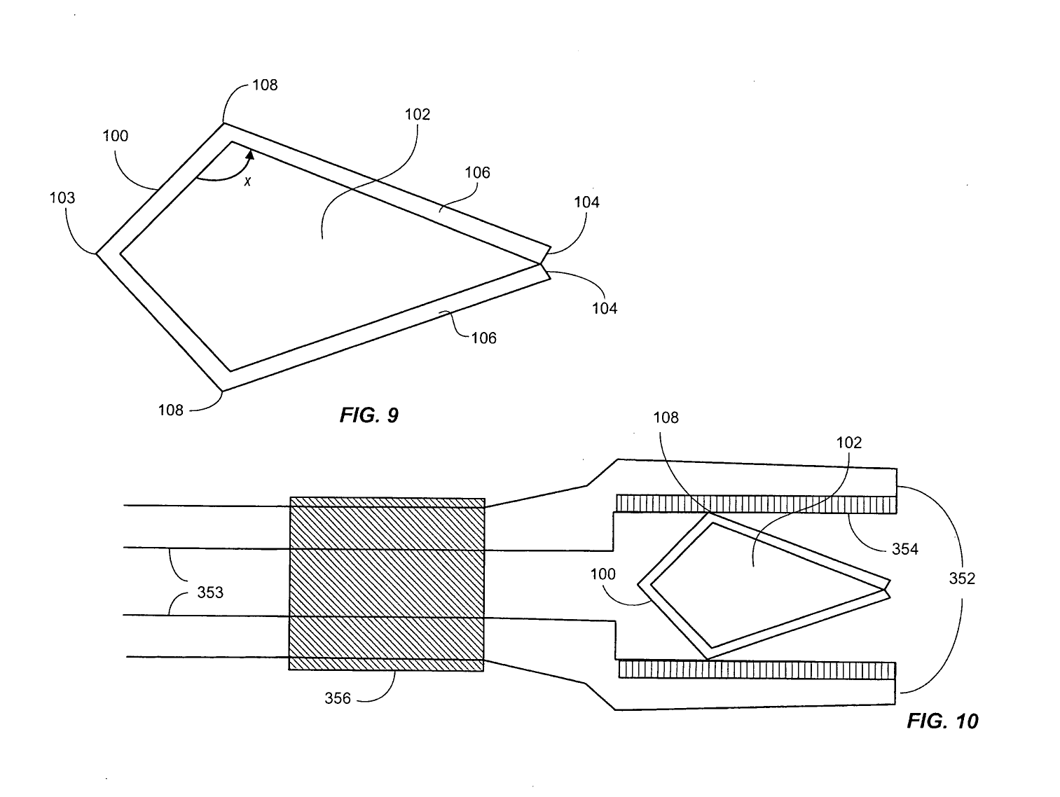

FIG. 8 is a partial side view of a multi-mode clip applier 300, schematically illustrating features of a hand-held body portion 302. Multi-mode clip applier 300 for example includes internal firing mechanisms controlled by an “open mode” switch or button (described below), to apply clips in the encircling or “open mode” configuration shown in FIG. 9. Referring briefly to FIG. 9, in the open mode configuration, a central portion 102 of clip 100 remains open, while the clip free ends 104 close together to encompass a tubular structure. Central portion 102 is for example formed by compressing clip 100, to bend clip arms 106 downward from clip shoulders 108, decreasing angle χ. Compare, for example angle χ of open mode staple 100, FIG. 9, to angle χ of un-clipped staple 100, FIG. 1. Multi-mode clip applier 300 may next, or alternately, compress clip 100 into closed or flattened position (see FIG. 4), for example by applying pressure to clip shoulders 108 to increase angle χ.

Returning to FIG. 8, hand-held body 302 of multi-mode clip applier 300 includes a fixed handle 304 and a trigger 306. Pulling trigger 306 toward handle 304, as indicated by directional arrow 308, compresses a clip held in the jaws of multi-mode clip applier into open mode, when mode switch 310 is enabled (See, e.g., FIG. 9). In one embodiment, trigger 306 extends within body 302 via a connected trigger plate 312. Upon compression of trigger 306 towards handle 304, trigger plate 312 for example rotates clockwise around a pivot point 314. A secondary plate 322 may connect with and rotate with trigger plate 312.

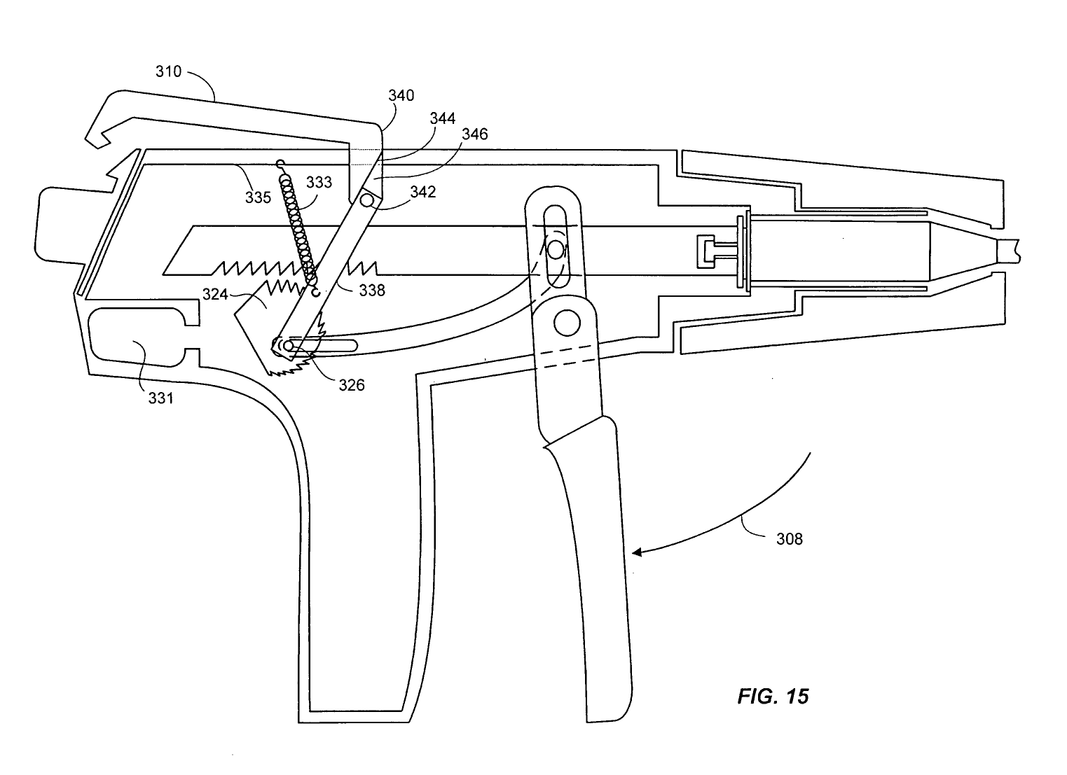

Secondary plate 322 includes a slot 320. When secondary plate 322 rotates clockwise, slot 320 angles such that a first rod, dowel or sliding bar 318 slides or rolls down slot 320. A connecting element 316, connected with first sliding bar 318, moves down and backwards (proximally) as bar 318 slides. Connecting element 316 in turn advances a cogwheel, gear or ratchet 324. Ratchet 324 is rotatably mounted with connecting element 316, for example riding on a second rod, dowel or sliding bar 326, within a second slot 328 of connecting element 316. When rotated, ratchet 324 engages a toothed rack 330. Teeth of ratchet 324 are for example at a positive engagement angle relative to the teeth of rack 330 due to the location of the ratchet pivot point (e.g., second sliding bar 326). When trigger 306 is pulled, trigger plate 312 rotates clockwise, first sliding rod 318 drops down slot 320 of secondary plate 322 and connecting element 316 moves proximally and downward, as allowed by connecting element 316 chamber 331 Connected ratchet 324 abuts a stop mechanism, for example a rear, inner wall 325 of body 302, which initiates clockwise rotation of ratchet 324, as indicated by arrow 327, to advance rack 330 distally (in the direction of arrow 332) and fire (e.g., apply clip 100 with) multi-mode applier 300. A return spring 333 is fixed at one end to a stationary structure or part of applier 300, for example to a spring mount (not shown) or to an inner wall 335 of body 302 itself. As shown in FIG. 15, return spring 333 acts between lever a 338 and an inner wall 335 of stationary structure to return lever 338 to the pre-firing position shown in FIG. 8. Lever 338 connects with ratchet 324, for example at second sliding bar 326, which serves as a pivot point for lever 338. Return motion of lever 338 rotates ratchet 324 clockwise (arrow 327) and into contact with rack 330.

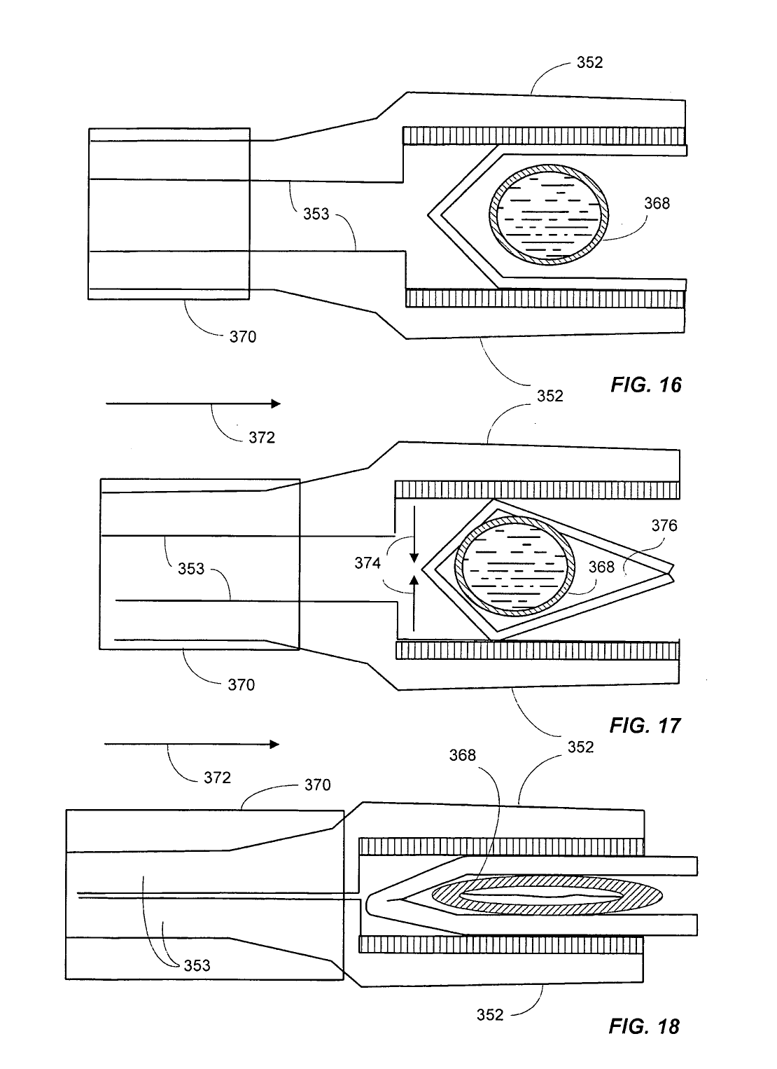

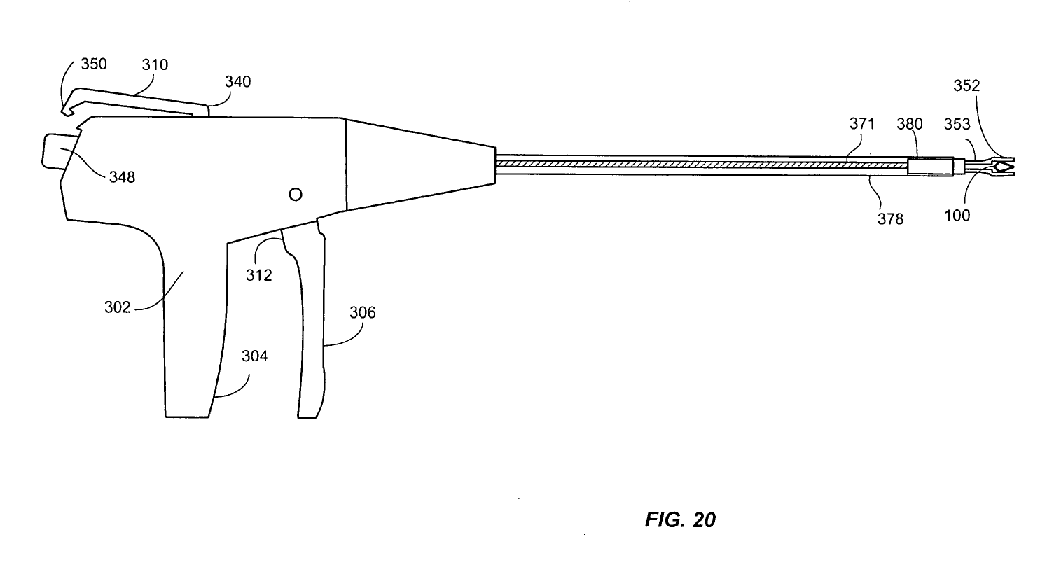

When trigger 306 is pulled back (arrow 308), rack 330 moves distally (arrow 332), for example compressing springs 334 to actuate a firing pin 336. Pin 336 may in turn advance a jaw actuator, such as a cannula (e.g., cannula 370, described with respect to FIGS. 16-19, below) or a wire (e.g., wire 371) over the distal jaws of multi-mode applier 300 (see FIGS. 19 and 20). Optionally, pin 336 advances a wire connected with, and configured for actuating, the distal jaws. When switch 310 is engaged, distal movement of components of multi-mode applier 300 are limited, e.g., by interaction between switch 310 and lever 338.

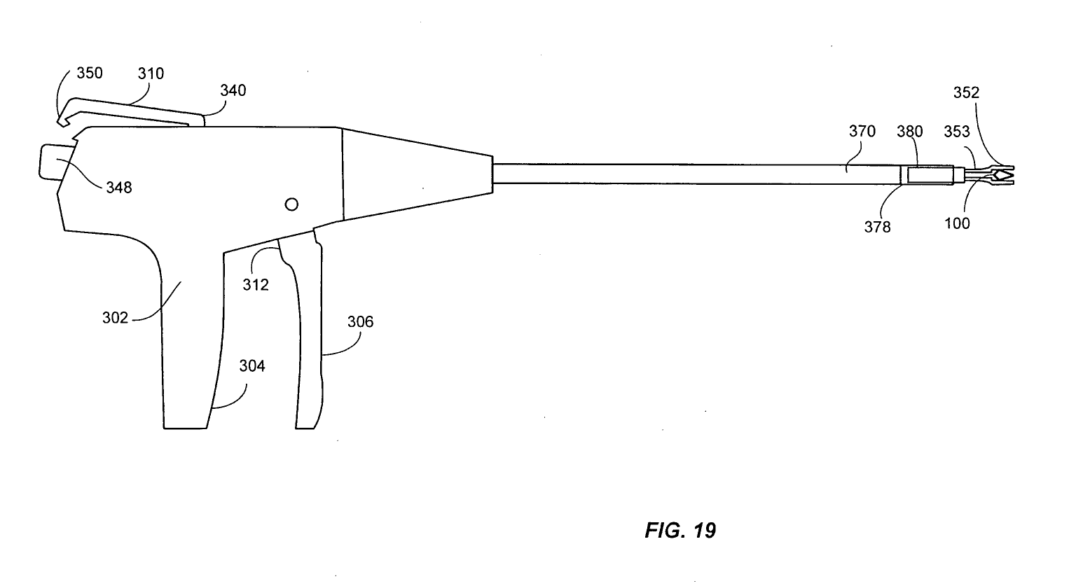

In one embodiment, lever 338 and ratchet 324 connect with connecting element 316 via second sliding bar 326. Connecting element 316 moves down and back, ratchet 324 abuts a stop such as rear inner wall 325, and rotates clockwise (arrow 327), engaging and advancing toothed rack 330. Second sliding bar 326 rides distally in slot 328 with rotation of ratchet 324, rotating lever 338 counterclockwise (arrow 339). Optionally, lever 338 rotatably connects with a distal end 340 of switch 310, at a lever pivot 342. Counterclockwise rotation of lever 338 is inhibited by a lever stop 344 at distal end 340 of switch 310. Lever stop 344 is for example an extrusion of switch 310, the border of an etched, lever accommodating portion 346, or a bar, rod or other inhibiting mechanism. When switch 310 is engaged, e.g., connected to a release button 348 via a proximal catch 350, lever 338 cannot move through its full counterclockwise. range of motion; thus, forward motion of ratchet 324 and toothed rack 330 are limited, as is advancement of the aforementioned cannula over the jaws of multi-mode applier 300. As shown in FIG. 10, jaw arms 353 angle upward proximally-to-distally, and thus, jaws 352 are forced closer together as the cannula advances distally over the jaw arms. Inhibiting distal advancement of the cannula therefore limits closure of applier jaws such that a clip 100 is not fully flattened, but rather compressed into open mode, as shown in FIG. 17. When release button 348 is activated, e.g., by pressing or sliding, proximal catch 350 disengages and releases switch 310. When released, switch 310 may pivot about lever pivot 342 when lever 338 abuts lever stop 344, and toothed rack 330, and thus the cannula, advance further distally, to further compress jaws 352 into flattening or closed-clip mode. See FIG. 18. Multi-mode clip applier 300 may include an automatic clip-feeding mechanism similar to prior-art clip feeders; however, the clip feeding mechanism in multi-mode applier 300 may be configured such that a new clip 100 does not load into jaws 352 until the jaws have moved through flattening mode. This for example allows a surgeon to place, verify and then close one clip at a time. In another embodiment, a feeder selection switch or button allows the surgeon to choose whether clips 100 are loaded after open-mode jaw closure, after closed-mode jaw closure or both. An additional feeder stop (not shown) is provided to inhibit feeding of clips after jaw closure, for example allowing the surgeon to place multiple clips in open mode, verify all clips, and then flatten all clips, without new clips loading each time the jaws move through flattening mode.

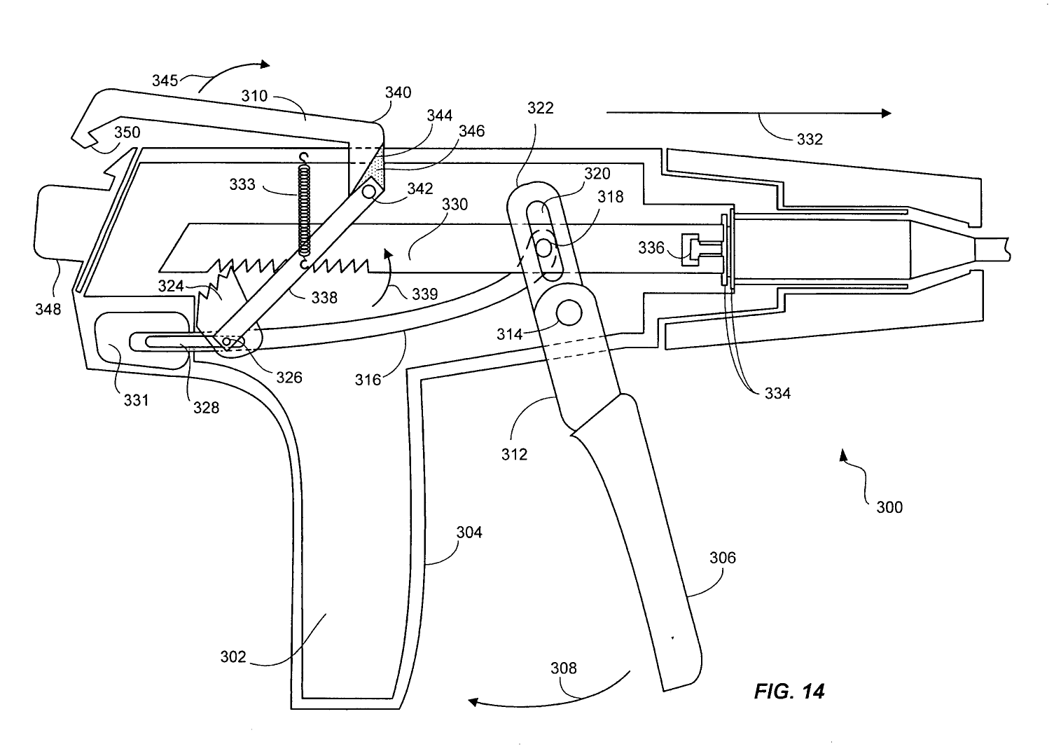

It will be appreciated that the mechanical firing mechanisms described with respect to FIG. 8 (and also with respect to FIGS. 14-15) may be replaced by electromechanical devices, such as a linear actuator that slides distally upon actuation via a user interface (e.g., trigger 308 or a button, which may replace the firing function of trigger 308), to press firing pin 336. A degree of pressure on firing pin 336 and/or a level of jaw 352 closure is for example selectable or programmable via the user interface.

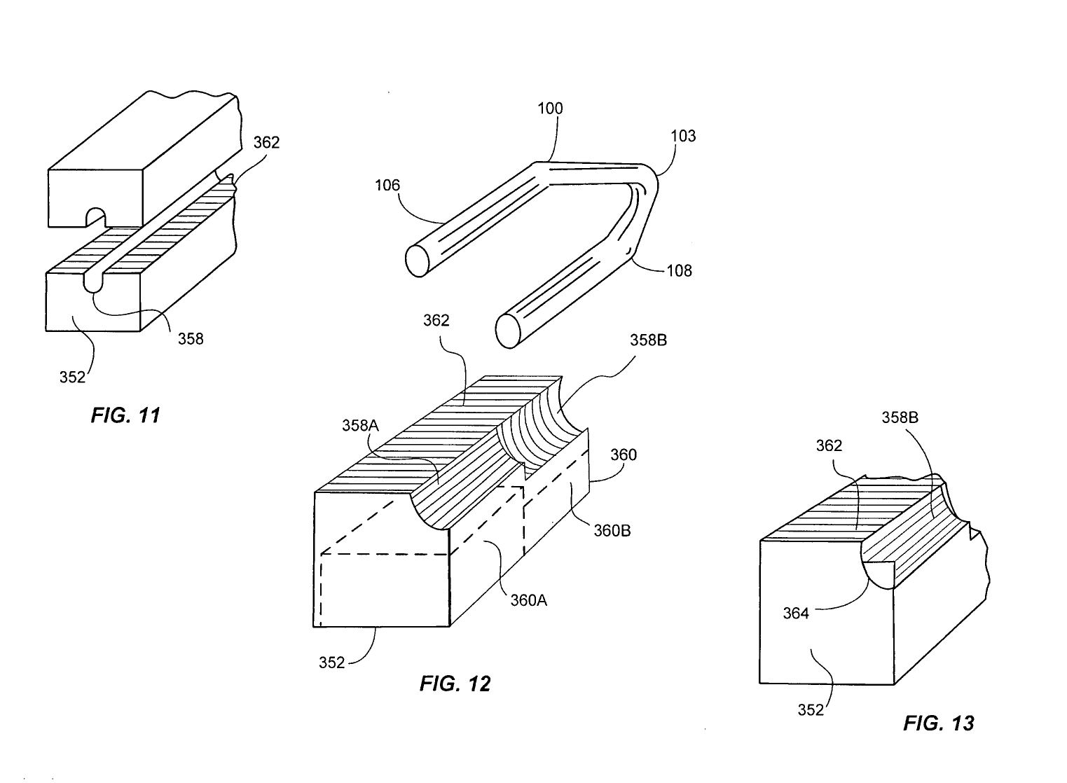

FIG. 10 shows jaws 352 of multi-mode applier 300, according to an embodiment. As shown, jaws 352 extend from jaw arms 353, bounded by a collar 356, which prevents jaws 352 from opening too far and releasing a held clip 100. Clip 100 has been compressed into open mode and is held by jaws 352, which for example employ a pair of gripping pads 354 to secure clip 100. Gripping pads 354 may be releasably attached, textured pads, or gripping pads 354 may be configured as textured portions, serrations or a series of extrusions and/or indentations of jaws 352. Alternately, as shown in FIG. 11, jaws 352 may include a channel 358 sunk within a clipping surface 362 of one or both jaws. Channel 358 may be sized to fit a selected clip size, and/or textured to aid in securely grasping clip 100. Jaw arms 353 angle upward, proximally-to-distally, such that as a jaw actuator (see, e.g., FIGS. 19-20) slides distally over arms 353, jaws 352 are forced closer and closer together.

Turning to FIG. 12, in one embodiment, jaw 352 includes a shallow channel portion 358A and a deep channel portion 358B. One or both of shallow and deep channel portions 358A, 358B are flexible and backed by inner supports 360, which lend additional strength to jaws 352. Supports 360 are for example dual-part supports connected with inner components of multi-mode clip applier body 302, moving medially toward jaw clipping surfaces 362 when trigger 306 is pulled. Supports 360 maybe separately controllable by selecting a clipping mode, allowing for separate compression of flexible, shallow channel 358A and deep channel 358B. When switch 310 is engaged, a first support part 360A, underlying shallow channel 358A, may be activated to compress shallow channel 358A. The range of motion provided when switch 310 is engaged advances a cannula distally over jaws 352 to a point sufficient to activate and compress first support part 360A. A clip 100 held with its arms 106 secured within shallow channel portion 358A, and its shoulder 108-to-nose 103 portion (see FIG. 1) protruding into deep channel portion 358B, is compressed into open mode (arms 106 are pressed to bring free ends 104 together) when switch 310 is engaged. When switch 310 is disabled (e.g., release button 348 is pressed), the cannula advances further distally, to a point sufficient to also activate and compress a second support part 360B underlying deep channel portion 358B, to fully flatten clip 100. As shown in FIG. 13, one or both jaws 352 may include a clip stop 364, to prevent clip 100 from sliding out of channel 358.

FIGS. 14 and 15 show multi-mode clip applier 300 with switch 310 released, prior to and after firing of trigger 306, respectively. With switch 310 disengaged, lever 338 rotates further in the direction of arrow 339 when trigger 306 is pulled, thus advancing a cannula or actuation wire further distally over jaws 352, to flatten clip 100 (see FIG. 18). Motion of switch 310 around lever pivot 342 may be inhibited only by the body 302 of the multi-mode clip applier. Lever 338 contacts lever stop 344 during its rotation; however, clip 310 is not fixed to release button 348 by proximal catch 350 and thus swings or pivots clockwise (arrow 345) around lever pivot 342 as lever stop 344 is pushed by lever 338. After firing, return spring 333 pulls lever 338 back to its pre-fire position (FIG. 14).

As shown in FIG. 14, the toothed section of rack 330 is longer than is required by the number of teeth on ratchet 324. Ratchet 324 may thus engage toothed rack 330 at a range of locations, providing for closure of clip 100 around a range of duct, vessel or tubular structure size, thickness and consistency. For example, ratchet 324 is set to engage toothed rack 330 proximally where a cannula must be advanced farther in the distal direction, to compress jaws 352 for clipping a small or hardy structure. On the other hand, ratchet 324 engages toothed rack 330 at a distal end of the toothed section, when less compression of jaws 352 is desired, for example when applier 300 is set (e.g., via external control) to clip a large or fragile structure.

FIGS. 16-18 illustrate multi-mode clipping of a duct. In FIG. 16, jaws 352, holding clip 100, are positioned about a duct 368. A cannula 370 is shown in resting position upon jaw arms 353. Open mode application is selected, for example by engaging switch 310, trigger 306 is fired (trigger 306 is pulled) and cannula 370 advances distally over jaw arms 353 and closes jaws 352 sufficiently to produce an open-mode clip 100, as in FIG. 17. Arrows 372 and 374 illustrate distal and medial motion of the cannula and jaws, respectively. Clip 100 is for example a radiopaqued clip or a clip bearing radiopaqued markers. After applying clip 100 in open mode, e.g., during laparoscopic cholecystectomy, a physician may image the surgical site to determine whether clip 100 is appropriately placed.

Open-mode application of imageable clips may enhance, or in some instances replace, traditional cholecystectomy. Rather than requiring a separate procedure (e.g., laparoscopic cholecystectomy) prior to clip placement, open-mode clips (e.g., FIG. 17) may be removably attached to ducts and imaged to insure correct placement, prior to flattening. If a misplaced clip is discovered, open-mode clips may be easily removed by inserting a tool or an applier jaw in open area 376, and freeing clip 100. This approach may reduce the need for separate, invasive cholecystectomy, eliminating minor cystic duct injuries that occur during this procedure.

Traditional cholangiography typically employs injectable contrast medium, which is slowly introduced into the biliary system the system (e.g., into the liver in percutaneous transhepatic cholangiography) to image the biliary tree and verify patient anatomy. During this procedure, small scissors are introduced into the abdominal cavity and used to make an incision in the cystic duct. The tip of the catheter is introduced into the incision and advanced down the duct. It should be noted that the catheter is often secured with a clip, and that care must be taken not to over crimp the catheter clip. This may be accomplished by having an assistant inject saline through the catheter as the clip is closed, and halting clipping just as resistance to flow is felt. However, such steps may not be necessary when multi-mode clip applier 300 is used to secure the catheter with an open-mode clip. Once the catheter is secured within the cystic duct, fluorescent fluids are injected to create contrasts that facilitate structural identification and diagnosis. The cholangiogram thus gives the surgeon a “map,” clarifying the relationship between the cystic duct, common bile duct, and the hepatic ducts. Although very helpful in preventing transection of the wrong internal structures, the traditional cholangiogram itself causes minor injury to the cystic duct. Utilizing open-mode clips as markers may reduce these injuries. It should be noted that prior art clip appliers preclude the use of clips as structural markers, since these clips can not be applied in an open mode.

Following imaging, for example by conventional x-ray, a properly placed open-mode clip 100 (FIG. 17) may be flattened to securely clamp the duct, as shown in FIG. 18. This is for example accomplished by pressing release button 348 to release switch 310, prior to firing applier 300. Cannula 370 is then allowed to advance to its distal-most position overjaw arms 353 upon firing, fully clamping jaws 352 over clip 310 and duct 368. In one embodiment, cannula 370 rides over collar 356.

FIG. 19 shows one embodiment of multi-mode clip applier 300. Cannula 370 is enclosed by a supportive sheath 378, and/or engages with a circumferential closure element 380. Cannula 370 pushes closure element 380 forward to compress jaws 352 for open mode clips 100, when switch 310 is engaged, or flattened clips 100, when switch 310 is released. In FIG. 20, cannula 370 is replaced by a jaw-actuating wire 371 riding within supportive sheath 378, for engaging closure element 380 and compressing jaws 352.

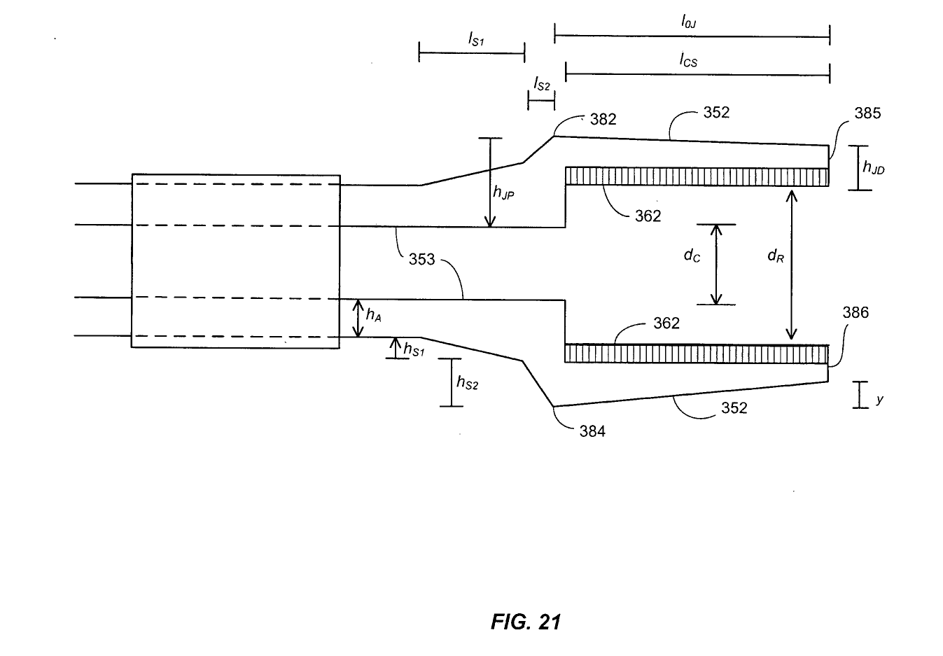

FIG. 21 shows exemplary dimensions of jaws and jaw arms 352, 353. Although dimensions may vary according to design preference and desired application, in one embodiment, clipping surfaces 362 have a length (l_{CS}) of about ½″. Jaws 352 have a proximal height (h_{JP}) of about 9/64″, tapering medially to a distal height (h_{JD}) of about 1/16″. Proximal height h_{JP} includes a first lateral slope height (h_{S1}) of about 1/32″, a second slope height (h_{S2}) of about 1/16″ and jaw arm height (h_{A}) of about 3/64″. Jaws 352 have a first lateral slope length (l_{S1}) of about 1/32″ (not shown to scale) and a second slope length (l_{S2}) of approximately 1/32″ . Jaws 352 have an outer jaw length (l_{OJ}) of about ¼″- 17/32″, from upper/lower proximal jaw points 382, 384 to upper/lower jaw ends 385, 386. Upper and lower proximal jaw points 382, 384 are vertically separated from clipping surfaces 362 by h_{JP}, as shown, and horizontally separated from clipping surfaces 362 by about 1/64″. The heighty from jaw point 382/384 to corresponding distal jaw end 385/386 is about 3/32″. When fully relaxed/unclamped, jaws 352 have a relaxed distance (d_{R}) between clipping surfaces 356 of about 3/16″. Fully clamped jaws may not exceed a clamped separation distance (d_{C}) of ⅛″. Jaws 352 compress clip 100 so as to strain the clip beyond a deformable yield point, to an extent that when the compressive forces are relaxed, the clip remains in a deformed (“open”) state that is only partially collapsed, as shown in FIG. 17. The full extent and range of motion of the jaws may be predetermined as a function of the materials properties for the clip.

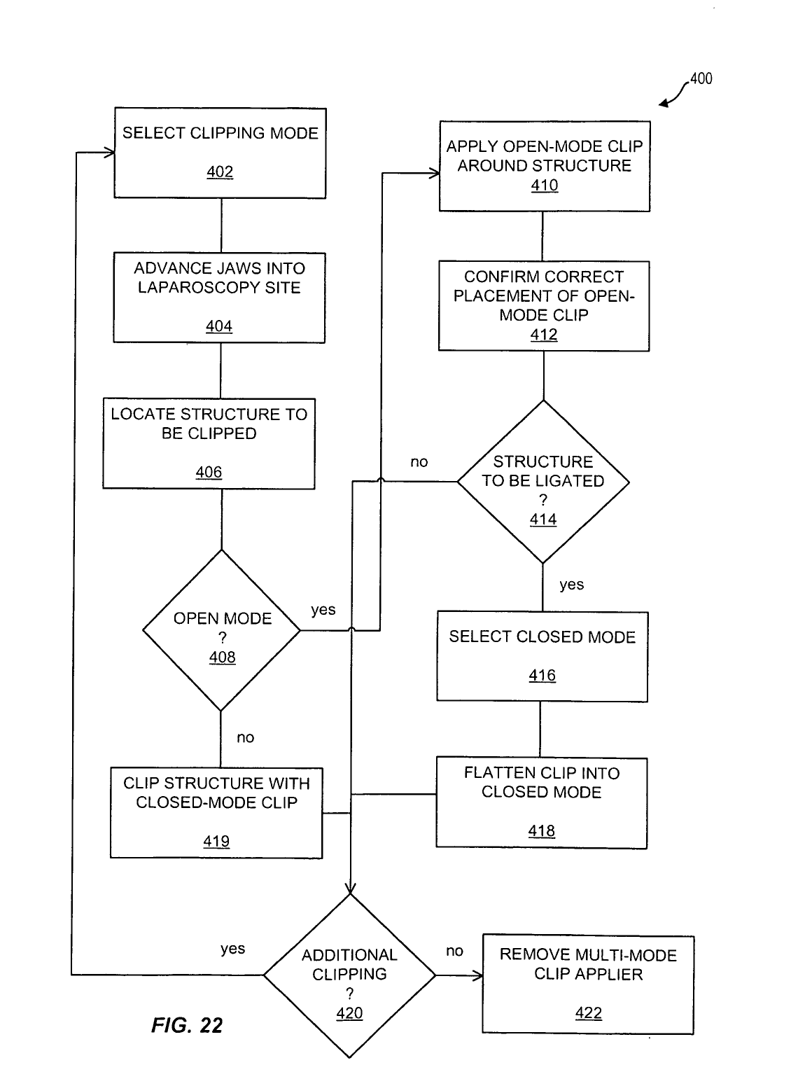

FIG. 22 shows an exemplary method 400 of use for multi-mode clip applier 300. In step 402, a practitioner, e.g., a surgeon or assistant, selects a clipping mode. The jaws of loaded multi-mode clip applier 300 are advanced into an access site, in step 404. In one example of steps 402, 404, the practitioner engages or disengages open-mode switch 310 to select the clipping mode of applier 300. Jaws 352 (at the distal end of cannula 370 and/or supportive sheath 378) are advanced into a patient's body cavity through a laparoscopy site. In step 406, The practitioner (e.g., surgeon) locates the structure to be clipped, for example through use of endoscopic cameras utilized with multi-mode clip applier 300. When the procedure being performed is a cholecystectomy, the surgeon for example locates the gallbladder and cystic duct and arteries. If open mode is selected, decision 408, one or more clips are applied in open mode around the located structures, in step 410. In one example of step 410, clip 100 is applied in open mode around the cystic duct, during cholecystectomy. In step 412, correct placement of the open-mode clip (or clips) is confirmed. In one example of step 412, when clip 100 includes radiopaqued markers, x-ray imaging is used to visualize clip 100 and confirm its placement around the desired body structure. If the clipped structure is to be ligated, decision 414, the surgeon selects closed mode, in step 416. The open mode clip is clamped into flattened, closed mode, in step 418. In one example of steps 416-418, the surgeon presses release button 348 to disengage open-mode switch 310. Disengaging open-mode switch 310 allows for increased closure of jaws 352, and upon actuation of trigger 306, the applied open-mode clip 100 (depicted in FIG. 17) is flattened into closed mode (depicted in FIG. 18). It will be appreciated that if multi-mode clip applier 300 is retracted from the clipping site or removed from the patient's body during imaging, it is advanced or re-introduced, and the jaws positioned about the open-mode clip, prior to closing the jaws to flatten the clip. In step 422, the multi-mode clip applier is removed from the patient's body, if further clipping is not required (decision 420).

Returning to decision 408, if open-mode clipping is not selected, a clip is applied in closed mode, in step 419. If no further clips are to be applied (decision 420), multi-mode clip applier is removed from the patient's body (step 422). Closed-mode clipping may be selected (without first clipping in open-mode) when the surgeon is certain that the correct structure is identified for clipping, for example, in open procedures, or in cases of marking or ligating an obviously identifiable structure, such as vessels surrounding a tumor or aneurism.

It will be understood by those skilled in the art that the multi-mode clip applier is described herein with exemplary clip firing elements. However, open-mode switch 310, its internal connections to firing mechanisms (e.g., cannula 370 and elements in communication therewith) and release button 348 may be utilized in connection with alternate mechanisms to effectuate clip firing and loading, for example as described in the aforementioned U.S. Pat. Nos. 5,171,249, 6,843,794 and 5,100,420.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A multi-mode clip applier for applying surgical clips, comprising:
a hand-held applier body having a handle, a trigger and distal jaws for holding a surgical clip therebetween, the jaws configured to selectively produce an open-mode surgical clip upon actuating the trigger.

2. The applier of claim 1, further comprising a jaw actuator and a firing pin for advancing the jaw actuator over arms of the distal jaws; wherein pressure upon the firing pin is reduced when open-mode application is selected, to reduce distal advancement of the actuator over the jaw arms.

3. The applier of claim 2, the jaw arms angling upward proximally-to-distally such that jaw closure increases as the jaw actuator advances distally over the jaw arms.

4. The applier of claim 1, further comprising an open-mode button, for selecting the open-mode surgical clips.

5. The applier of claim 4, wherein the jaws produce a closed-mode surgical clip when the open-mode button is deselected.

6. The applier of claim 1, further comprising a linear actuator for advancing the firing pin upon actuation of the trigger.

7. Apparatus for applying surgical clips, comprising:
a hand-held applier body having a handle, a trigger, distal jaws for holding a surgical clip therebetween, and a jaw actuator for actuating closure of the jaws; and
an open-mode switch, for limiting closure of the distal jaws to selectively produce an open-mode surgical clip, when the trigger is actuated.

8. Apparatus of claim 7, wherein actuating the trigger when the open-mode switch is selected limits distal advancement of the jaw actuator overjaw arms of the distal jaws, to limit jaw closure.

9. Apparatus of claim 7, further comprising a release button for releasing the open-mode switch, to produce a closed surgical clip upon actuating the trigger.

10. Apparatus of claim 9, wherein the jaw actuator advances to a most distal position over the jaw arms when the open-mode switch is released and the trigger is actuated, to close the jaws and flatten the surgical clip.

11. Apparatus of claim 7, wherein the jaws hold opposing arms of the surgical clip, the jaws closing upon the opposing arms when the trigger is actuated, to bend the arms together at opposing clip free ends.

12. Apparatus of claim 7, the jaw actuator selected from a cannula and a wire.

13. Apparatus of claim 12, the jaw actuator forcing a closure element distally over opposing arms of the jaws, to bend the surgical clip into the open mode.

14. A multi-mode clip applier for applying surgical clips, comprising:
a hand-held applier body having a handle, a trigger, distal jaws for holding a surgical clip therebetween, and a jaw actuator for actuating closure of the jaws;
an open-mode switch for selectively limiting closure of the distal jaws, to produce an open-mode surgical clip upon actuating the trigger; and
a release button for releasing the open-mode switch, to produce a closed surgical clip upon actuating the trigger.

14. The applier of claim 13, the jaw actuator selected from a cannula and a wire, wherein actuation of the firing mechanisms pushes the jaw actuator distally to close the jaws when the open-mode switch is released, or partially close the jaws when the open-mode switch is engaged.

15. The applier of claim 14, further comprising a jaw closure element; the jaw actuator pushing the jaw closure element distally over angled jaw arms when the trigger is actuated, to close or partially close the jaws, as a function of the open-mode switch.

21. The applier of claim 19, further comprising a lever configured with the ratchet at a first lever end and with the open-mode switch at a second lever end; the lever abutting the open-mode switch when the open-mode switch is engaged, to limit clockwise rotation of the ratchet when the trigger is pulled.

22. Method for multi-mode surgical clipping, comprising:
from a hand-held applier which grasps a surgical clip between its jaws, forcing the surgical clip into an open-mode configuration; and
if desired, forcing the open-mode surgical clip into a closed surgical clip.

23. Method of claim 23, wherein forcing comprises actuating a trigger when holding a handle of the applier.

24. Method of claim 23, wherein forcing the clip into a closed surgical clip comprises releasing an open-mode switch configured with the hand-held applier.

25. Method for multi-mode surgical clipping, comprising:
selecting an open clipping mode on a multi-mode clip applier;
advancing distal jaws of the multi-mode clip applier to a body structure; and
clipping the structure with an open-mode clip, to circumferentially bound the structure;
verifying correct placement of the open-mode clip;
selecting a closed clipping mode on the multi-mode clip applier, when the placement of the open-mode clip is correct; and
flattening the clip into closed mode with the multi-mode clip applier, when the placement of the open-mode clip is correct.
